(19) United States
(12) Patent Application Publication
(10) Pub. No.: US 2007/0197926 A1
(43) Pub. Date: Aug. 23, 2007

(54) Method for analysing an intracardiac electrocardiogram and an electrophysiological system as well as a computer program product

(76) Inventors: Kenneth Danehorn, Vaxholm (SE); Peter Harmat, Lund (SE); Patrik Milton, Malmo (SE); Luping Pang, Erlangen (DE); Martin Stridh, Hjarup (SE); Prof. Leif Sornmo, Lund (SE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/649,616

(22) Filed: Jan. 4, 2007

(30) Foreign Application Priority Data
Jan. 13, 2006 (EP) 06000737.4

(51) Int. Cl. A61B 5/04

(52) U.S. Cl. 600/509

(57) ABSTRACT

The present invention relates to a method for analysing an intracardiac electrocardiogram to identify at least one of the A wave, V wave and H wave on at least one of the electrogram signals, comprising the steps of pre-processing the electrogram signal; calculating an adaptive threshold for the A, V or H wave, wherein the adaptive threshold depends on the noise level of the electrogram signal and on the type of wave; and identifying the A, V or H wave by searching the electrogram signal within a time window determined e.g. from the position of another wave on the same or another electrogram signal.

### CROSS REFERENCE TO RELATED APPLICATIONS

This application claims priority of European application No. 06000737.4 filed Jan. 13, 2006, which is incorporated by reference herein in its entirety.

### FIELD OF THE INVENTION

The present invention relates to a method for analysing an intracardiac electrocardiogram (IECG) consisting of one or several electrogram signals each acquired by a catheter placed inside the heart, in order to identify at least one of the A wave, V wave and H wave on at least one of the electrogram signals. The invention also relates to a electrophysiological system adapted for performing the method, and a computer program product allowing the method to be performed by a computer.

### BACKGROUND OF THE INVENTION

Electrophysiological studies (EPS) are used for diagnosing defects in the heart's conduction system. EPS is carried out by inserting catheters into e.g. the femoral, subclavian, internal jugular or antecubital veins, so they can reach places inside the heart near the sinus node, atrioventricular (AV) node, bundle of His or the ventricles. The catheters can record electrogram signals as well as apply electrical stimulation pulses to stimulate a specific region of the heart. Different diagnostic programs of EPS are defined according to the stimulation pattern and the evaluation.

A typical distribution of catheters inside a human heart is illustrated in FIG. 1. FIG. 1 shows a four chamber view of the heart with particular focus on the electrical conduction system. Important components of the electrical conduction system of the heart include the sinus node 2, the AV node 4, the bundle of His 6 and the Purkinje fibres 8. The electric activity of a heart beat generally starts at the sinus node 2, which rhythmically initiates 70-80 impulses per minute without any nerve stimulation. Depolarisation then propagates through the atrial myocardium and causes the two atria to contract simultaneously. When the impulses reach the AV node 4, they are conducted more slowly. Thereafter, the electrical discharge travels rapidly to the bundle of His, which conducts the impulses to both ventricles, causing ventricular contraction.

The figure further shows a typical set of catheter positions consisting of a high right atrial catheter HRA, a coronary sinus catheter CS, a right ventricular apex catheter RVA and a His bundle catheter HIS.

The signals recorded by these catheters generally show a ventricular potential or V wave, as well as the atrial potential or A wave. The His bundle potential or H wave is generally recorded mainly by the His bundle catheter.

For the diagnosis of heart defects causing arrhythmias, it is generally desirable to be able to measure the time intervals between the different cardiac potentials, such as the A-H interval or the H-V interval.

During an IECG, an ordinary body surface electrocardiogram may also be acquired. Such a BSECG lead V1 recording is shown on the top of FIG. 2. The bottom signal of FIG. 2 is a His bundle electrogram (HIS) with the AV potential (A), His bundle potential (H) and ventricular potential (V) identified.

### SUMMARY OF THE INVENTION

It is an object of the invention to provide a method which can automatically identify all visible cardiac activations (A, H or V waves) on an intracardiac electrocardiogram.

This object is achieved by the method as well as by the electrophysiological system and the computer program product according to the claims.

The method also uses a simultaneously acquired body surface electrocardiogram (BSECG) and comprises the following steps:

Pre-processing the electrogram signal; identifying the QRS-complex in the body surface electrocardiogram; calculating an adaptive threshold for the A, V or H wave, wherein the adaptive threshold depends on the noise level of the electrogram signal and on the type of wave; identifying the A, V or H wave by searching the electrogram signal within a time window determined either from the position of the QRS complex in the BSECG, or the previously determined position of a wave on the same or another electrogram signal, and detecting the A, V or H wave within the time window using the adaptive threshold.

The BSECG is recorded as known in the art. For example, it may be a 12-lead BSECG providing the signals I, II, III, aVL, aVS, aVR and V1 to V6. Procedures for identifying the QRS complex on such BSECG is also known in the art and will not be further described. It is also possible, and may be used in an embodiment of the invention, to detect the P wave in the BSECG.

The number of electrogram signals within the IECG depends on the diagnostic protocol. Since the automatic wave detection of the present invention especially adapted to the His bundle signal, a HIS signal will generally be present. The CS, HRA and RVA or further signals may be available according to the placement of the catheters.

The criteria to identify the A, V or H waves are based on the sequence of the waves appearing under different conditions and their amplitude relationship. Therefore, empirical searching time windows together with adaptive thresholds are used.

Adaptive thresholds mean that the thresholds are set according to the noise level within the electrogram signal. The time windows may be determined either from the position of the QRS complex in the BSECG, or from the position of a wave which has been previously identified on the same or a different electrogram signal.

According to a preferred embodiment, the BSECG is also used to define an R-R interval between two subsequent R waves. Thereby, it is possible to divide the electrogram signals into R-R intervals each corresponding to one heart beat.

According to another aspect of the invention, the classifiycation method is divided into different procedural branches, depending on the position of the catheters, the number of stimulation pulses (if any) and the type of the stimulation. The advantage of such branch definition is that the method can be better adapted to the various applications in an EPS and achieve more accurate results. In order to select the procedural branch, the number of stimulation pulses within the R-R interval is checked. Preferably, this is done on a stimulation marker signal, which is supplied by the EPS system generating the stimulation pulses. In the Siemens cardiac system “SENSIS”, this signal is called the “Stim” signal. According to the number of stimulation pulses and the type of the stimulation (antegrade, retrograde), one of several procedural branches may be followed.

According to a preferred embodiment, the pre-processing comprises: applying a high-pass or band stop filter on the electrogram signal, applying a non-linear transformation on the filtered signal in order to extract an envelope of the filtered signal, and applying a low-pass filter on the envelope of the filtered signal. Thereby, it is possible to obtain a positive-valued, smooth signal suitable for further analysis.

In case that stimulation pulses are applied to the heart during the acquisition of the IECG, the method may provide for removal of possible pacing artefacts. Preferably, this is achieved by detecting the ascending or descending edge of a stimulation wave on a stimulation marker signal, such as the “Stim” signal, and setting the electrogram signal to zero within a pre-determined time window around the detected ascending or descending edge.

Preferably, the analysis method is applied on an electrogram signal acquired by a catheter placed near the bundle of His (the HIS signal), and optionally at least one of several further electrogram signals are acquired by catheters placed in the right atrium (the HRA signal), in the coronary sinus (the CS signal), or near the right ventricular apex (the RVA signal).

An example procedural branch for identifying the A wave, H wave and V wave in the HIS signal and the A wave and V wave on the HRA or CS signal is defined in one of the claims. In case only a HIS signal needs to be analysed, this procedural branch may be reduced to the steps relating to the HIS signal. This procedural branch is preferably used when no stimulation pulses are used. Other procedural branches having, for example, different adaptive thresholds and different empirical windows, may be used under other conditions, for example during antegrade or retrograde stimulation. Examples for such different procedural branches shall be given below.

According to a further preferred embodiment, the adaptive thresholds are calculated after pre-processing of the signals. However, additional thresholds values may also be calculated from the signals before pre-processing and used to confirm that a certain threshold is reached.

The invention is further directed to an electrophysiological system comprising a data analysis station, which is adapted for performing the above method, and to a computer program product containing program code which, when installed on a computer, will allow the computer to perform the above method.

### BRIEF DESCRIPTION OF THE DRAWINGS

Examples and preferred embodiments of the invention shall now be described in detail with reference to the accompanying drawings, in which

FIG. 1 is a cross-sectional view of a human heart;

FIG. 2 shows an exemplary body surface ECG recorded on lead V1, and an intracardiac ECG recorded with the HIS bundle electrode;

FIG. 3 is a flow diagram illustrating the main steps of an analysis method according to an embodiment of the invention;

FIG. 4 is a flow diagram illustrating the steps of pre-processing;

FIG. 5 is a flow diagram illustrating the step of the non-linear transform of FIG. 4 in more detail;

FIG. 6 illustrates the effects of pre-processing on a HIS signal, where A is the raw signal, B is the high-pass filtered signal, C is the filtered signal after non-linear transformation and D is the low-pass filtered signal of C;

FIG. 7 is a flow diagram showing the steps of an embodiment of a procedural branch;

FIG. 8 shows a body surface ECG and the intracardiac ECG consisting of HIS, HRA and RVA signals.

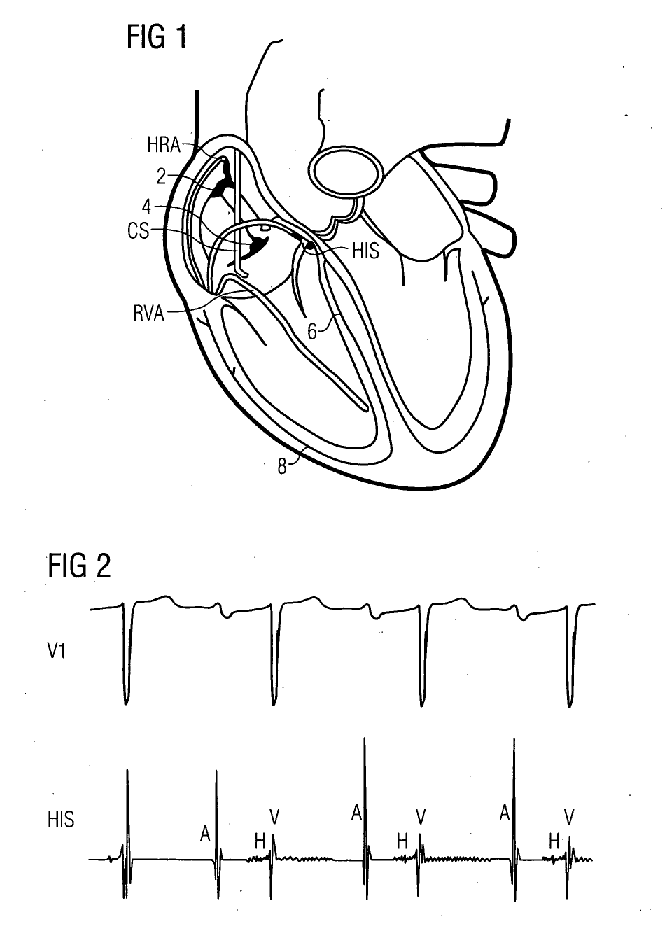

### DETAILED DESCRIPTION OF THE INVENTION

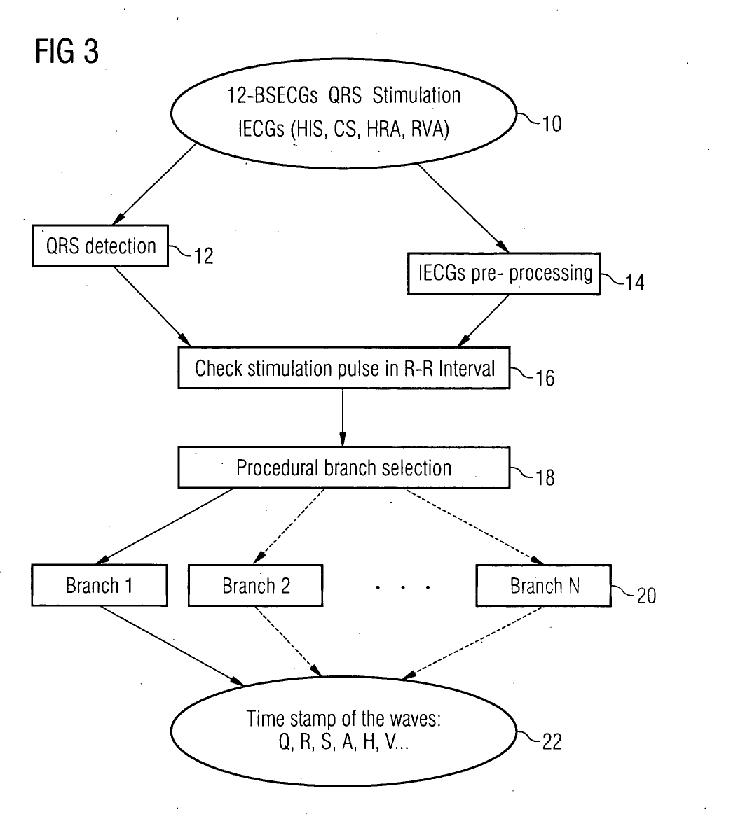

FIG. 3 gives an overview of a method according to an embodiment of the invention. Box 10 contains the input signals from the EPS system, for example the Siemens “SENSIS” system. In this example, this is a 12-lead body surface ECG, as well as an intracardiac ECG containing HIS, CS, HRA and RVA electrogram signals. In addition, the EPS system also furnishes a stimulation marker signal containing information about any external stimulation to the heart, such as the “Stim” signal.

The body surface ECG is then used for QRS detection by means of any available method 12. A pre-processing is applied to the IECG signals in step 14.

The QRS detection is used to divide each intracardiac electrogram signal into R-R intervals. In step 16, one or several selected R-R intervals are checked for stimulation pulses. This information is used to select one of several procedural branches 20 in step 18, but may also be used to determine whether a pacing artefact removal must be performed.

Hence, for the actual classification of the A, H and V waves, the method automatically divides into one of N procedural branches, depending on the presence and number of stimulation pulses, and on the type of available signals. After one of the algorithm branches is completed, the detected waves are provided with a time stamp indicating their type, i.e. A, H, V etc. and the results are outputted to the EPS system in step 22.

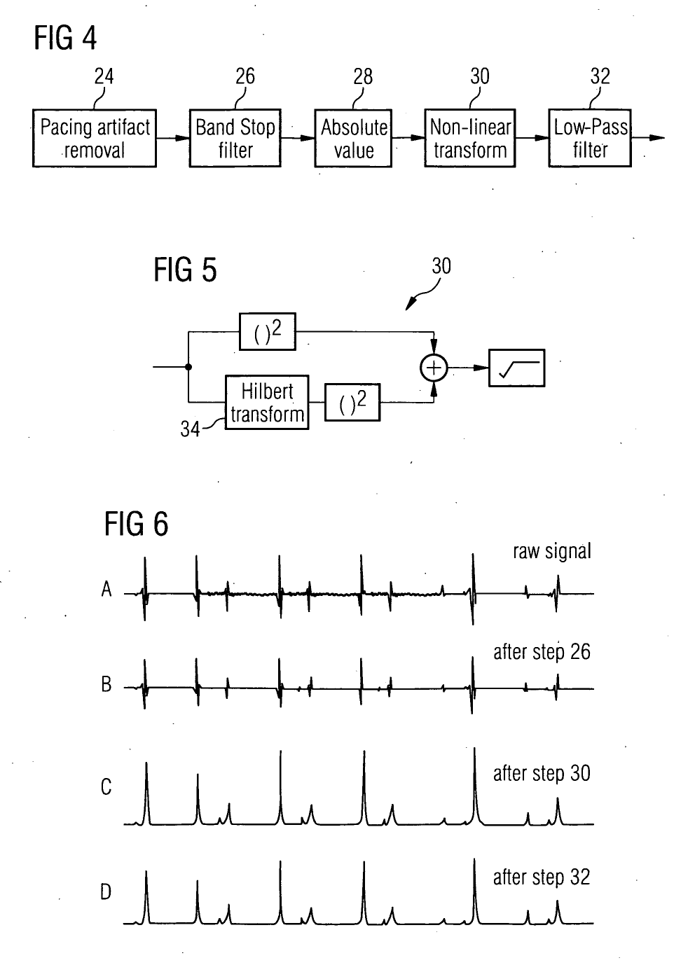

Some of the steps illustrated in FIG. 3 shall be described in greater detail in the following. Starting with the pre-processing step 14, reference is made to FIG. 4 showing the pre-processing in greater detail. According to FIG. 4, the pacing artefact removal 24 is performed before filtering of the signal, though in other embodiments it may be performed after the other pre-processing steps.

If a stimulation pulse is sent to one electrode of a catheter, e.g. the HRA 12 catheter, this may cause distortion of the other electrogram signals, which is called “pacing artefact”. To remove such artefact, a stimulation marker signal from the EPS system is used, which indicates each stimulation pulse as a short, preferably square stimulation wave. This stimulation wave may be detected for example by its ascending edge. Due to the nature of the stimulation marker signal, there is a 10 ms time delay between the beginning of a pacing artefact and the ascending edge of the stimulation wave. Besides, it is supposed that the width of the pacing artefact is not larger than 20-30 ms. Therefore, a 10-30 ms, preferably 20 ms window is selected around the ascending edge and the effected IECG signals (HIS, HRA, RVA or CS) are set to zero within this window.

The first step 26 of the pre-processor is a linear, time invariant band stop filter, designed to suppress ringing noise in the base line, see signal A in FIG. 6. The band stop filter ideally suppresses all frequencies between about 30 and 70 Hz. The cut-off frequencies should be selected without loss of any important clinical information, and without significant effect on the wave form. Instead of the band stop filter, a high-pass filter with a cut-off frequency of around 70 Hz may also be used.

In step 28, the absolute value of the filtered signal is taken. Next, a non-linear transformation is performed in step 30. This step is designed to calculate a deterministic, positive-valued signal y(n), referred to as the envelope of the filtered signal x(n).

The details of step 30 are illustrated in FIG. 5. A linear, time-invariant filter known as the Hilbert transform is used. The effect of the steps in FIG. 5 may be written as
y(n)=|x(n)|+2/n|x(n+1l)−x(n−1)|

Due to the calculated difference between x(n+1) and x(n−1), the Hilbert transform amplifies high gradients and thereby produces easily detectable peaks.

Finally, a low-pass filter with a cut-off frequency of approximately 40 Hz is applied in step 32. The result of this filter is shown as signal D in FIG. 6.

For performing the wave identification in one of the N branches, the electrogram signals are first sub-divided into R-R intervals by means of the BSECG. Whenever a new QRS complex is detected in the BSECG signal (V1 or another lead), the time window between the last two QRS complexes (R-R interval) will be focussed on to perform the wave identification in the IECG signals and their envelope signals.

In order to define the procedural branch, the number of the stimulation pulses within the R-R interval is checked in the “Stim” signal. According to the number of stimulation pulses and the type of stimulation (antegrade, retrograde), the algorithm will for example go to one of the following N=5 branches to identify the A, V and H wave in the IECG. Of course, further branches may be added, or some of the branches omitted, according to the individual requirements.

Branch 1—No stimulation pulse in the R-R interval, one catheter is placed in high right atrial or coronary sinus to get the HRA or CS signal.

Branch 2—One antegrade stimulation pulse in the R-R interval, the stimulation is placed either in the HRA or CS signal.

Branch 3—Two antegrade stimulation pulses in the R-R interval, the stimulation is placed either in the HRA or CS signal.

Branch 4—One retrograde stimulation pulse in the R-R interval, the stimulation is placed either in the RVA signal.

Branch 5—No stimulation pulse in the R-R interval, only the HIS signal is acquired; no catheter is placed in high right atrial or coronary sinus.

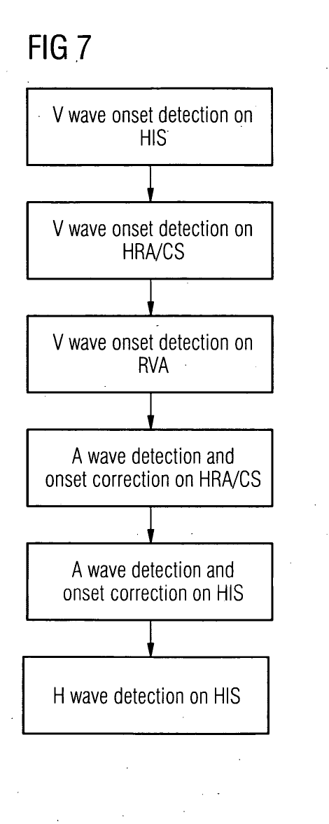

The main steps of branch 1 are illustrated in FIG. 7. The other branches may be modifications of this branch. According to FIG. 7, the V wave onset is first detected on the HIS signal, then on the HRA or CS signal and finally on the RVA signal. Then the A wave is searched in the CS/HRA, and then the HIS signal; H wave detection in the HIS signal is the last step.

Each detection step uses a windowing technique, where the position of the window is determined based on the results of a previous detection step and the width of the window is generally pre-determined and empirically developed.

Within these windows, the filtered signal is searched to find either the time point where the signal crosses a certain adaptive threshold, which is defined as the onset of a wave, or the window is searched for a maximum, which must also be above a certain threshold.

Adaptive thresholds mean that the thresholds are set according to the estimation of the average noise level (baseline) in the filtered IECG signals. IECG signals have a time-variant signal to noise ratio, so that the average noise level should be calculated for each R-R interval. In a preferred embodiment, the R-R interval is first divided into 8 sub-segments; then the local maximums are calculated for each sub-segment. The minimal local maximum value is regarded as the baseline or noise level in this signal segment.

The adaptive thresholds for the different wave detections are written as:
Thr_Φ_X=α* Baseline_X
where X is the IECG signal (such as HIS, HRA, CS or RVA) or the respective envelope signal, Φ is the type of wave to be detected (A, V or H) and α is a pre-determined value, preferably an integer value, which is empirically determined.

In the following, examples are given for 5 different procedural classification branches, which are chosen according to the above list.

According to requirements, either a threshold on the pre-processed envelope signal, or on the raw signal, may be used in the different procedural branches.

In the following examples, the indication V_HIS indicates the detected position of the V wave onset on the HIS signal, A_CS indicates the A wave onset in the CS signal, etc.

Branch 1

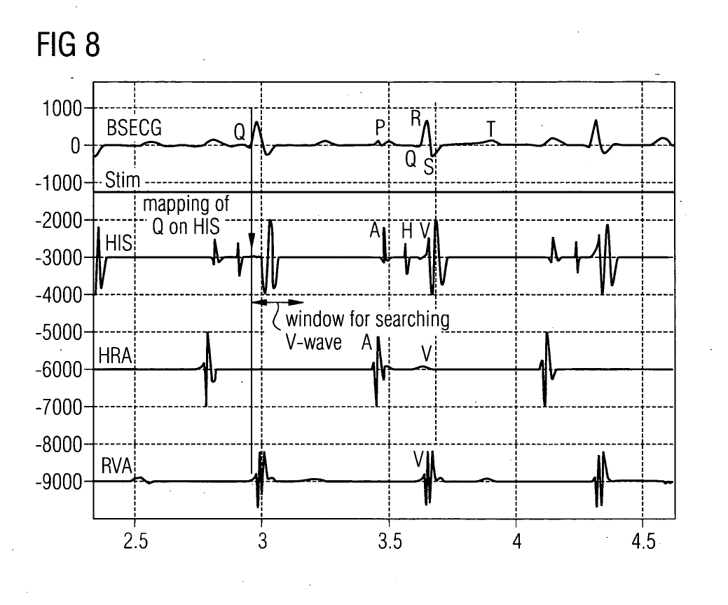

The source signals of branch 1 are the BSECG II, HIS, and HRA or CS signals. No stimulation pulse exists in the current R-R interval (FIG. 8).

1. V wave onset detection in HIS (V_HIS): To define the time window, the position of the Q wave is taken or “mapped” from the BSECG onto the HIS signal. If the mapping point on the HIS signal is over the V wave threshold Thr_V_HIS, the onset of the V wave is backward searched in the HIS signal within a 30 ms window. Otherwise, the onset of the V wave is forward searched in the filtered HIS signal until the signal amplitude is over the threshold Thr_V_HIS.

2. V wave onset detection in HRA or CS (V_HRA/CS): The detected V wave onset on HIS signal is mapped to the HRA or CS signal. The V wave maximum in the HRA or CS is searched in a 100 ms window after the mapping point. Then, the V wave onset in HRA or CS is backward corrected in a 30 ms window using the threshold Thr_V_HRA/CS.

3. V wave onset detection in RVA (V_RVA): The V wave maximum in the RVA is searched within the 100 ms around the mapping point from the R wave in the BSECG. Its onset is also backward corrected in a 30 ms window using the threshold Thr_V_RVA.

4. A wave detection in the HRA or CS (A_HRA/CS): In the HRA or CS, the A wave has larger potential so that it can be more easily detected. So the A wave is first detected in the HRA or CS signal. The maximum value is searched between the first V wave +80 ms to the second V wave onset in the R-R interval. If this maximum is over the threshold Thr_A_HRA/CS, it is detected as A wave in the HRA or CS. Thr_A_HRA is larger then Thr_A_CS because the A wave potential is mostly higher in the HRA signal than in the CS signal. Once the A wave is confirmed in the HRA or CS signal, its onset is corrected by a threshold of 1/10 of the A wave maximum in the signal. If no A wave is found in the HRA or CS signal, no A wave is searched in the HIS signal.

5. A wave detection in the HIS (A_HIS): The window [A_HRA/CS−50 ms: A_HRA/CS+60 ms] is applied to the HIS signal. The maximum within this window is detected as the A wave in the HIS signal. The onset correction of the A wave consists of two steps: first, it is backward corrected by the threshold Thr_A_HIS in a 20 ms window in the HIS signal; second, a forward correction is done on the filtered HIS signal by the threshold Thr_A_HIS.

6. Multi-A waves detection: A multi-A waves detection is additionally considered for specific arrhythmia conditions (e.g., atrial flutter). If the first A wave is found in the R-R interval, other local peaks will be further searched in the two windows: [A_HRA/CS+100 ms : V_HRA/CS−80 ms] and [Last_V_HRA/CS +100 ms: A_HRA/CS−80 ms]. If the local peak is over 60% of the first A wave amplitude in the HRA or CS signal, it is detected as another A wave. Then the corresponding multi-A waves are further searched in the HIS signal.

7. H wave detection in the HIS (H_HIS): The window for searching the H wave in the HIS signal depends on the position of A and V wave and their distance. If no A wave is detected, H wave is searched in a fixed window [V_HIS−60 ms : V_HIS−15 ms]. If A wave is detected, the beginning of the window is adjusted according to the distance between the V wave onset and the A wave, that is, whether the AV interval is larger than 150 ms. In the window, the local peak that is over the threshold Thr_H_HIS is detected as H wave. If there is no local peak, the maximal value that is over the same threshold is considered as H wave.

Branch 2

In this branch, one antegrade stimulation pulse is placed in the R-R interval, which indicates the existence of the signal HRA or CS.

1. V wave onset detection in HIS: V wave onset detection has the same process as that in branch 1.

2. V wave onset detection in HRA or CS: The detected V wave onset on HIS signal is mapped to the HRA or CS signal. The V wave maximum in the HRA or CS is searched in a 60 ms window after the mapping point.

3. V wave onset detection in RVA: The V wave maximum in the RVA will be searched within the 50 ms around the mapping

point from the R wave. Its onset is also backward corrected in a 60 ms window using the threshold Thr_V_RVA.

4. A, H wave detection: The placement of the stimulation pulse plays an important role for the A wave detection. According to the distance between the stimulation pulse and the current V wave onset, two sub-branches are considered for the A, H detection. Sub-branch 1 is applied if the distance between the stimulation pulse and the current V wave onset is larger than 200 ms. The same H wave detection as in branch 1 is also used here. Sub-branch 2 is applied if the distance between the stimulation pulse and the current V wave onset is smaller than 200 ms.

Branch 3

The difference between the branch 3 and branch 2 is that two antegrade stimulation pulses can occur in the R-R interval, e.g. during the fast continuous pacing or gradual decrease of the pacing interval (e.g., Wenckbach point analysis). In many cases, the second antegrade pacing leads to the coincidence of the atrial and ventricular excitation, which makes it difficult to separate the A, H and V waves. Therefore, the time window for searching the A wave after the first pacing is defined as [Stimulation pulse1+20 ms: Stimulation pulse2−20 ms], while the end of the window should not be closer than 150 ms to the V wave onset. The maximum in this time window that is over the threshold is detected as A wave in the HRA or CS signal. Then, the corresponding A wave will be further detected in the HIS signal. The beginning of the window in the HIS signal is the middle position between the first stimulation pulse and the A_HRA/CS; the end of the window is 50 ms after the A_HRA/CS but 80 ms before the V_HIS.

Branch 4

In this branch, a retrograde stimulation pulse is placed in the R-R interval in the RVA signal. The retrograde pacing causes inversed cardiac conduction from ventricular to atrial. That is, the wave sequence after the stimulation pulse is first a V wave and then an A wave in the normal case. The V wave and A wave can often coincide, which enhances the difficulty to detect the A wave in the HIS signal.

1. V wave detection in HIS and HRA/CS: The position of the Q wave is considered as that of the V wave.

2. V wave detection in RVA: Because the pacing is placed in the RVA signal, the V wave will not be earlier than 10 ms after the stimulation pulse.

3. A wave detection in HRA/CS: Due to the retrograde conduction, the A wave in the current R-R interval is induced by the retrograde stimulation pulse in the last R-R interval. The window of searching the A wave in HRA or CS signal begins from 120 ms after the first V wave in the current R-R interval. The end of the window is 50 ms before the stimulation pulse in the current R-R interval. If the maximum in this window is over the threshold Thr_A_HRA/CS, then it is considered as A wave and its onset is backward corrected in a 30 ms window.

4. A wave detection in HIS: The window [A_HRA/CS−30 ms: A_HRA/CS+50 ms] is applied to further search the maximum in the HIS signal. The maximum is detected as the A wave in the HIS signal. Its onset is also backward corrected in a 30 ms window.

5. Spontaneous A, H, V wave detection after the last retrograde stimulation pulse. It is mentioned that more attention should be paid to the last retrograde stimulation pulse. After the retrograde conduction induced by the last stimulation pulse, a spontaneous wave sequence can be followed. The identification of the spontaneous waves is the same as that explained in the branch 1.

Branch 5

Comparing to branch 1, only the HIS signal is acquired for the wave identification. No stimulation pulse is placed in the R-R interval. It is not easy to separate the A and H wave in the HIS signal without any other reference signal. As a solution, the detection of the P wave in the body surface ECG signal II is used as the reference to define the window of the A wave detection.

1. V wave onset detection in HIS: The same procedure is applied as in branch 1.

2. A wave detection in the HIS: The window [P onset: P onset+60 ms] is applied to search the maximum in the HIS signal. The maximum is detected as the A wave in the HIS signal. The onset correction of the A wave consists of two steps: first, it is backward corrected by the threshold Thr_A_HIS in a 30 ms window in the HIS signal; second, a forward correction is done on the filtered HIS signal.

3. H wave detection in the HIS: The same procedure is applied as in branch 1.

1.-11. (canceled)

12. A method for analysing an intracardiac electrocardiogram of a patient to identify a wave on an electrogram signal of the intracardiac electrocardiogram, comprising:
acquiring the electrogram signal by placing a catheter inside a heart of the patient;
simultaneously acquiring a body surface electrocardiogram of the patient;
pre-processing the electrogram signal;
identifying an QRS-complex in the body surface electrocardiogram;
calculating an adaptive threshold for the wave depending on a noise level of the electrogram signal and a type of the wave;
identifying the wave by searching the electrogram signal within a time window; and
detecting the wave within the time window using the adaptive threshold.

13. The method as claimed in claim 12, wherein the time window is determined from a position of the QRS complex in the body surface electrocardiogram or from a previously determined position of the wave on the same or another electrogram signal.

14. The method as claimed in claim 12, wherein the wave is selected from the group consisting of: A wave, V wave, and H wave.

15. The method as claimed in claim 14, wherein in order to identify at least two of the A wave, V wave, and H wave on the electrogram signal of the intracardiac electrocardiogram, the method further comprising:
defining an R-R interval between two subsequent R waves on the body surface electrocardiogram,
checking the R-R interval for a presence frequency of stimulation pulses, and
selecting one of a plurality of procedural branches for identifying the A wave, H wave and V wave depending on a position of the catheter, the presence frequency of stimulation pulses and a type of stimulation.

16. The method as claimed in claim 12, wherein the catheter placed inside the heart of the patient is selected from the group consisting of: a HRA catheter, a CS catheter, a RVA catheter, and a HIS catheter.

17. The method as claimed in claim 16, wherein the electrogram signal is a HIS signal acquired by the HIS catheter placed near bundles of His.

18. The method as claimed in claim 17, wherein a further electrogram signal is acquired by the HRA catheter placed in a right atrium, or the CS catheter placed in a coronary sinus, or the RVA catheter placed near a right ventricular apex.

19. The method as claimed in claim 18, wherein a first procedural branch for identifying an A wave, a H wave and a V wave on the HIS signal and the A wave and the V wave on the HRA or CS signal is defined by:
defining an R-R interval between two subsequent R waves on the body surface electrocardiogram,
calculating adaptive thresholds for the V wave, A wave and H wave in each of the HIS, HRA or CS and RVA signals depending on a wave type and a noise level of the respective signal in the R-R interval,
identifying the V wave in the HIS signal by searching the signal within a first time window determined from a position of the QRS complex and detecting the V wave using the respective adaptive threshold,
identifying the V wave in the HRA or CS signal by searching the signal within a second time window determined from the detected onset of the V wave in the HIS signal and detecting the V wave using the respective adaptive threshold,
identifying the A wave in the HRA or CS signal by searching the signal within a third time window determined from the detected onset of the V wave in the HIS signal and detecting the A wave using the respective adaptive threshold,
if the A wave is detected on the HRA or CS signal, identifying the A wave in the HIS signal by searching the signal within a fourth time window determined from the detected onset of the A wave in the HRA or CS signal and detecting the A wave using the respective adaptive threshold, and
identifying the H wave in the HIS signal by searching the signal within a fifth time window determined from the detected position of the A wave and the V wave and a distance of the A wave and the V wave in the HIS signal and detecting the H wave using the respective adaptive threshold.

20. The method as claimed in claim 19, wherein the adaptive thresholds are calculated from the HIS, HRA or CS and RVA signals after pre-processing and additional thresholds are calculated from the HIS, HRA or CS and RVA signals before pre-processing.

21. The method as claimed in claim 19, wherein the first procedural branch is selected when no stimulation pulses are detected in the R-R interval.

22. The method as claimed in claim 19, wherein a further procedural branch is selected when an antegrade stimulation is detected in the R-R interval and is a variation of the first procedural branch.

23. The method as claimed in claim 12, wherein the pre-processing step comprises:
applying a high-pass or band stop filter on the electrogram signal,
applying a nonlinear transformation on the filtered signal in order to extract an envelope of the filtered signal, and
applying a low-pass filter on the envelope of the filtered signal.

24. The method as claimed in claim 23, wherein the nonlinear transformation is a Hilbert transformation.

25. The method as claimed in claim 23, wherein the pre-processing step further comprises removing a possible pacing artefact resulting from a stimulation pulse from the electrogram signal.

26. The method as claimed in claim 25, wherein the possible pacing artefact is removed by:
detecting an ascending or descending edge of a stimulation wave on a stimulation marker signal, and
setting the electrogram signal to zero within a predetermined time window around the detected ascending or descending edge.

27. The method as claimed in claim 26, wherein the predetermined time window is in a range of 10 ms to 30 ms.

28. An electrophysiological system for performing an intracardiac electrocardiogram for a patient, comprising:
a catheter inserted into a heart of the patient that acquires an electrogram signal of the intracardiac electrocardiogram;
a lead that acquires a body surface electrocardiogram;
a signal processor that amplifies and filters the electrogram signal; and
a data analysis station that:
pre-processes the electrogram signal,
identifies an QRS-complex in the body surface electrocardiogram, calculates an adaptive threshold for a wave depending on a noise level of the electrogram signal and a type of the wave,
identifies the wave by searching the electrogram signal within a time window, and
detects the wave within the time window using the adaptive threshold.

29. A computer program for analysing an intracardiac electrocardiogram of a patient to identify a wave on an electrogram signal of the intracardiac electrocardiogram acquired by a catheter placed inside a heart of the patient, comprising:
a sub computer program that pre-processes the electrogram signal;
a sub computer program that identifies an QRS-complex in a body surface electrocardiogram;
a sub computer program that calculates an adaptive threshold for a wave depending on a noise level of the electrogram signal and a type of the wave;
a sub computer program that identifies the wave by searching the electrogram signal within a time window; and
a sub computer program that detects the wave within the time window using the adaptive threshold.

30. The computer program as claimed in the claim 29, wherein the wave is selected from the group consisting of: A wave, V wave, and H wave.

31. The computer program as claimed in the claim 30, further comprising:
a sub computer program that defines an R-R interval between two subsequent R waves on the body surface electrocardiogram,
a sub computer program that checks the R-R interval for a frequency of stimulation pulses, and
a sub computer program that selects one of a plurality of procedural branches for identifying the A wave, the H wave and the V wave depending on a position of the catheter, the frequency of stimulation pulses and a type of stimulation.
